(19) United States
(12) Patent Application Publication
(10) Pub. No.: US 2017/0252301 A1
(43) Pub. Date: Sep. 7, 2017

(54) MG STEARATE-BASED COMPOSITE NANOPARTICLES, METHODS OF PREPARATION AND APPLICATIONS

(71) Applicants: Andreas VOIGT, Berlin (DE); Sonja LEHMANN, Berlin (DE); Annette BA-ZANDT, Berlin (DE); Therakine BioDelivery GmbH, Berlin (DE)

(72) Inventors: Andreas Voigt, Berlin (DE); Sonja Lehmann, Berlin (DE); Annette Assogba-Zandt, Berlin (DE)

(21) Appl. No.: 15/517,973

(22) PCT Filed: Oct. 8, 2015
(86) PCT No.: PCT/US15/54725

§ 371 Date: Apr. 10, 2017

Related U.S. Application Data
(60) Provisional application No. 62/062,212, filed on Oct. 10, 2014.

(51) Int. Cl. A61K 9/14; C07K 16/00

(52) U.S. Cl. CPC A61K 9/145; C07K 16/00

(57) ABSTRACT

Disclosed are biocompatible composite nanoparticles and methods of preparing biocompatible composite nanoparticles. Also disclosed ate composite nanoparticles which are biocompatible, biodegradable and have numerous other advantages, and also examples of preparation of the nanoparticles and applications for intracellular delivery.

### CROSS-REFERENCE TO RELATED APPLICATIONS

The present application is a national stage entry according to 35 U.S.C. §371 of PCT Application No. PCT/US2015/054725 filed on Oct. 8, 2015, which claims priority to U.S. Provisional Application Ser. No. 62/062,212 filed on Oct. 10, 2014.

### TECHNICAL FIELD

The subject matter herein generally relates to biocompatible composite nanoparticles comprising magnesium stearate nanoparticles and at least one oil.

### BACKGROUND

Biomedicine would benefit tremendously from nanoparticulate carriers that can effectively provide intracellular delivery and targeted delivery of active agents. Conventional approaches have failed to achieve or create nanoparticulate carriers that reliably and effectively provide such intracellular and targeted delivery. Therefore, there is an ongoing need in the field for such nano-particulate carriers. One important goal for any new biocompatible composite nanoparticle is that the nanoparticle be able to provide a number of advantageous properties.

### SUMMARY

Various embodiments are described here, and do not limit the scope of the invention in any way.

According to an embodiment, a biocompatible composite nanoparticle is prepared.

As further described herein, and according to an embodiment, a biocompatible composite nanoparticle is created that has a magnesium stearate-oil base.

In at least one embodiment, the composite nanoparticles provides several advantageous and surprisingly beneficial properties; these properties include, but are not limited to, biodegradability, biocompatibility, complex payload capabilities (for instance, carrying passive and active ingredients, magnetite, fluorescent marker), control of size, design of the surface composition of the nanoparticles for control of interaction with tissue (e.g., interaction with exposed functional groups, antibodies, peptides, receptors), control of uptake into cells, protection of active ingredients, efficiency of active ingredient function, control of targeting or accumulation at target site (e.g. upon intracellular sustained delivery of the active ingredients), and any combination thereof.

In at least one embodiment, the sustained intracellular release effect of the nanoparticle is increased compared to conventional carriers. Other carriers may include, but are not limited to, complexes, viruses, liposomes, and solid lipid nanoparticles.

According to another embodiment, an essentially hydrophilic payload (i.e. one or more hydrophilic active ingredients) is incorporated into an essentially hydrophobic magnesium stearate-oil based nanoparticle.

According to another embodiment, at least one oil is mixed with magnesium stearate to create a paste-like composition. In at least one embodiment the paste-like composition is low in water and oil fractions. The paste-like composition is added to a plant oil and the system is stirred to achieve a special particle size distribution. In at least one embodiment, the hydrophobic system is supportive and prevents excessive phase separation.

According to an embodiment, the nanoparticles formed may be essentially separated from the oil by a series of established procedures. In an embodiment, the established procedures may include filtration, sedimentation, centrifugation, magnetic separation, washing, or any combination thereof.

In at least one embodiment, the composite nanoparticles are functional for use in intracellular delivery of one or more active ingredients.

In at least one embodiment, the composite nanoparticles are functional for use in targeted delivery of one or more active ingredients.

Those skilled in the art will recognize additional features and advantages upon reading the following detailed description, and upon viewing the accompanying drawings.

### BRIEF DESCRIPTION OF THE DRAWINGS

Certain embodiments will now be described, by way of example only, with reference to the attached figures.

FIG. 1 shows representative results of a size-measurement of magnesium stearate nanoparticles; and

FIG. 2 is a representative set of size-measurement data.

### DETAILED DESCRIPTION

The following language and descriptions of non-limiting embodiments are set forth in order to provide a thorough understanding of the embodiments described herein. However, it will be understood by those of ordinary skill in the art that no limitations of the present embodiments are intended, and that further alterations, modifications, and applications of the principles of the present embodiments are also included.

Non-limiting embodiments are directed to biocompatible composite nanoparticles. Additional non-limiting embodiments are directed to composite nanoparticles which are biocompatible, biodegradable and which may possess superparamagnetic properties. Moreover, other non-limiting embodiments are directed to preparation and application of such composite nanoparticles for intracellular delivery and target delivery of a payload.

In one non-limiting embodiment, a composite nanoparticle is constructed based on MgStearate/oil as the main passive ingredient. MgStearate is not soluble in water and can be prepared from water-soluble NaStearate by addition of MgCl₂. This opens up a second method of preparation of MgStearate nanoparticles.

According to an embodiment, preparation of the composite nanoparticles may include an incorporation of only a fraction of hydrophilic components (for example, active ingredients, marker or supportive passive ingredients) into the MgStearate/oil based nanoparticles. These methods of preparation surprisingly produce composite nanoparticles with a number of advantages.

The active ingredients and functional ingredients may be any of a wide variety of agents, which are known to those skilled in the art. Examples of active ingredients and functional ingredients that can be used include, but are not limited to, proteins, peptides, nucleic acids, lipids, amino acids, carbohydrates and derivatives of these aforementioned ingredients, as well as conventional pharmaceutical active ingredients, magnetite, and fluorescent markers.

Non-limiting examples of active ingredients may be or include, but are not limited to, a protein, a humanized monoclonal antibody, a human monoclonal antibody, a chimeric antibody, an immunoglobulin, fragment, derivative or fraction thereof, a synthetic, semi-synthetic or biosynthetic substance mimicking immunoglobulins or fractions thereof, an antigen binding protein or fragment thereof, a fusion protein or peptide or fragment thereof, a receptor antagonist, an antiangiogenic compound, an intracellular signaling inhibitor, a peptide with a molecular mass equal to or higher than 3 kDa, a ribonucleic acid (RNA), a deoxyribonucleic acid (DNA), a plasmid, a peptide nucleic acid (PNA), a steroid, a corticosteroid, an adrenocorticostatic, an antibiotic, an antidepressant, an antimycotic, a [beta]-adrenolytic, an androgen or antiandrogen, an antianemic, an anabolic, an anesthetic, an analeptic, an antiallergic, an antiarrhythmic, an antiarterosclerotic, an antibiotic, an antifibrinolytic, an anticonvulsive, an anti-inflammatory drug, an anticholinergic, an antihistamine, an antihypertensive, an antihypotensive, an anticoagulant, an antiseptic, an antihemorrhagic, an antimyasthenic, an antiphlogistic, an antipyretic, a beta-receptor antagonist, a calcium channel antagonist, a cell differentiation factor, a chemokine, a chemotherapeutic, a co-enzyme, a cytotoxic agent, a prodrug of a cytotoxic agent, a cytostatic, an enzyme and its synthetic or biosynthetic analogue, a glucocorticoid, a growth factor, a hemostatic, a hormone and its synthetic or biosynthetic analogue, an immunosuppressant, an immunostimulant, a mitogen, a physiological or pharmacological inhibitor of mitogens, a mineralocorticoid, a muscle relaxant, a narcotic, a neurotransmitter, a precursor of neurotransmitter, an oligonucleotide, a peptide, a (para)-sympathomimetic, a (para)-sympatholytic, a sedating agent, a spasmolytic, a vasoconstrictor, a vasodilator, a vector, a virus, a virus-like particle, a virustatic, a wound healing substance and a combination thereof.

Non-limiting examples of passive ingredients and/or formulation ingredients may be or include, but are not limited to, MgStearate, NaStearate, metallic soaps, soaps, MgCl₂, Cetyl Palmitate, suitable plant oils, castor oil, and water.

The oil(s) may be any of a wide variety of agents, which are known to those skilled in the art. Suitable oils may include, but are not limited to, tocopherol, castor oil, plant oil, and any suitable oil accepted in biomedicine or cosmetics.

One of the surprising advantages achieved with the composite nanoparticles is the sustained intracellular release effect. This sustained intracellular release effect is in contrast to conventional carriers (e.g., complexes, viruses, liposomes, solid lipid nanoparticles) which lack the surprising benefits, since conventional carriers provide a rather instantaneous release.

The incorporation of hydrophilic payload into the hydrophobic MgStearate/oil based composite nanoparticle can be achieved via different routes.

For the purpose of this specification, the term “mixing” is intended to describe, for instance, a mechanical process or a mechanical treatment of the components. For example, mixing can comprise repeated cycles of pressing and folding or comparable processing steps,. which lead to an intense compression of the components.

MgStearate may be mixed with one or more ingredients (one ingredient is essentially an oil, for example, tocopherol or castor oil). The kind of mixing performed depends on the ingredient properties. Dry ingredients (for example, lyophilized proteins) have to be treated differently as compared to ingredients which are dissolved/dispersed in an aqueous medium (for example, magnetite nanoparticles or another protein preparation). The aim of this first formulation step is to obtain a paste-like composition with rather low water and oil fractions.

In a non-limiting embodiment, the paste-like composition is then added to a plant oil (or another type of oil that is accepted in biomedicine or cosmetics as a formulation medium). Thereafter, the system is stirred. Depending on the intensity and duration of stirring (in general, on the rheological parameters) a desired particle size distribution of the MgStearate/oil-based composite particles is generated. The rheological parameters permit one to obtain the desired nanoparticles when the parameters are adequately selected. Interestingly, it has been observed that a rather low stirring intensity provides a nanoparticle size of a few hundred nanometers. This is caused by surfactant properties of the main passive ingredients.

The hydrophobic medium (plant or another permitted oil) is supportive to prevent an excessive phase separation of the components constituting the composite particles. The hydrophobic medium also functions to drive the MgStearate basic ingredient to form the particle side of the phase boundary particle/oil, thus separating the other ingredients more or less from the continuous oil phase as bulk.

According to another non-limiting method, active and passive ingredients may be added to a NaStearate solution. This mixture is concentrated to form a paste--like consistency. This multi-component paste is dispersed in plant oil with no extra surfactants (in addition to NaStearate). The system is stirred to transfer the paste into a highly dispersed phase distributed in the continuous oil phase. Thereafter, an amount of concentrated MgCl₂ solution is added, corresponding to a quantitative transformation of NaStearate into MgStearate. At appropriate rheological conditions again composite nanoparticles of a MgStearate basis are formed.

The nanoparticles can be essentially separated from the oil by a combination of established procedures (for instance, filtration, sedimentation, centrifugation, magnetic separation, washing etc.).

After separation from the oily base and transfer into an aqueous medium, the nanoparticles are ready for application or further chemical or physico-chemical treatment (for example, functionalization of the surface).

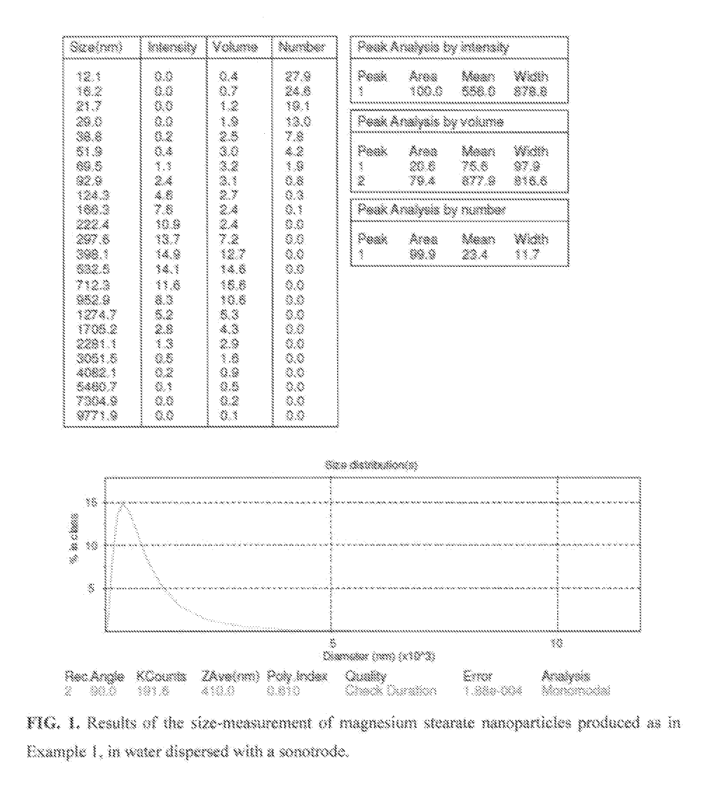

It has been unexpectedly found that the low energy input of a magnetic stirrer alone provides a nanoparticle suspension. A representative example of a particle size distribution (ZetaSizer) is shown in FIG. 1.

By means of a mechanical stirrer (for example, Heidolph RZR 2051) the energy input can be increased by an order of magnitude or even more. This can be used to produce desired changes in the nanoparticle size distribution.

It has been unexpectedly found that the nanoparticles offer a number of advantages. These advantages include, but are not limited to, nanoparticles that provide sustained delivery of active ingredients (i.e. payload), as well as reliable and reproducible intracellular delivery and targeted delivery of active ingredients.

Additional advantages include, but are not limited to, a combination of advantageous nanoparticle properties, including biodegradability, biocompatibility, multi-component composition, and optimum surface design of the nanoparticles sustained release of active ingredients. In the following, specific examples are described. These are merely examples, and shall not limit the scope in any way.

### EXAMPLE 1

0.75 g of sodium stearate and 0.08 g dry IgG selection are mixed to form a fine-grained powder. Water is added until a paste-like composition is formed. 30 mL of soy bean oil is then added to the paste-like composition and the resulting mixture is stirred using a magnetic stirrer at 850 rpm for 30 minutes. After stirring, 0.4 g MgCl₂ is added to the mixture and the system is stirred for an additional 45 minutes. The dispersion is then run through a centrifuge at 5000 rpm, for 10 minutes to separate out a particle fraction. For example, a centrifuge that may be used is the HERMLE Z 233 M-2 centrifuge. The system is then transferred to an aqueous environment.

The MgStearate-IgG composite nanoparticles exhibit a broad range of average particle diameter. The majority of MgStearate-IgG composite nanoparticles have average diameters ranging from approximately 150 nm to approximately 1000 nm. A magnetic stirrer can be used to create a nanoparticle suspension, the low energy input alone provides such a suspension. For example, a representative particle size distribution is shown in FIG. 1. In particular, FIG. 1 shows the results of the size-measurement of magnesium stearate nanoparticles produced as in Example 1, in water dispersed with a sonotrode.

### EXAMPLE 2

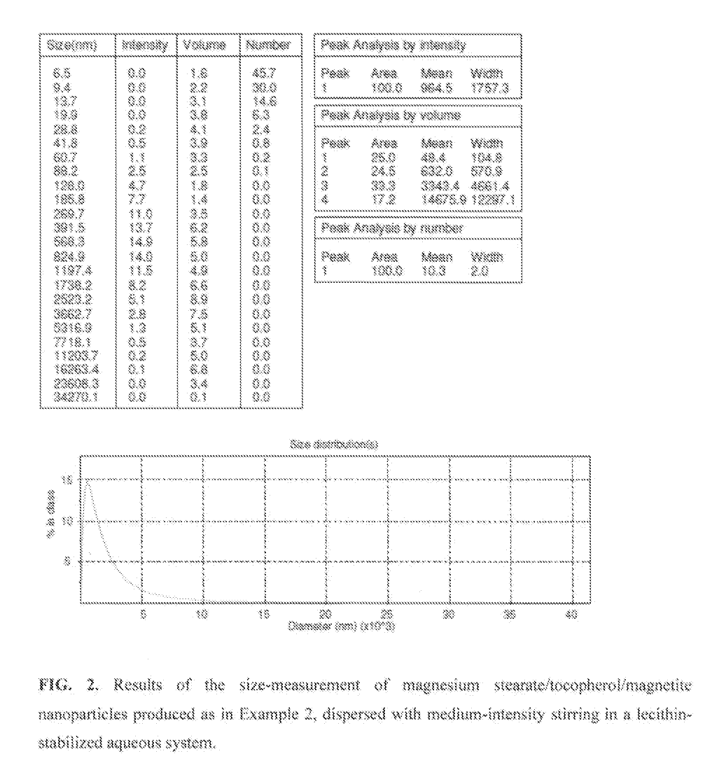

1 g of MgStearate, 0.2 g of tocopherol, 0.1 g dry IgG selection and 1 g of magnetite suspension are mechanically mixed to create a paste-like system. The paste-like system is then transferred to 20 mL of soy bean oil. The system is stirred at 1200 rpm for 2 hours. The resulting composite nanoparticles in an oil-based solvent system may be separated by a magnetic field of a permanent magnet. For instance, magnesium stearate/tocopherol/magnetite nanoparticles in oil may be separated by a magnetic field of a permanent magnet. FIG. 2 shows the results of the size-measurement of magnesium stearate/tocopherol/magnetite nanoparticles produced as in Example 2, dispersed with medium-intensity stirring in a lecithin-stabilized aqueous system.

According to one embodiment, particles (for instance, magnesium stearate/tocopherol/magnetite microparticles) prepared at low stirring intensity in an aqueous system and stabilized by lecithin are of microparticle size. Increase of stirring intensity results in particles of nanoparticle size.

A mechanical stirrer may be used to increase the energy input by an order of magnitude or more. For example, a mechanical stirrer which may be used is the Heidolph RZR 2051 mechanical stirrer. This process decreases the particle size into the nanoparticle size range. The MgStearate-IgG-tocopherol-magnetite composite nanoparticles exhibit a broad range of average particle diameter. The majority of MgStearate-IgG-tocopherol-magnetite composite nanoparticles have average diameters ranging from approximately 150 nm to approximately 1750 nm.

The embodiments shown and described herein are only examples, and do not limit the scope of the embodiments in any way.

What is claimed is:

1. A method of preparing one or more magnesium stearate based nanoparticles, comprising:
mixing at least one oil with magnesium stearate based nanoparticles to create a paste-like composition; and
separating the magnesium stearate based nanoparticles from the oil.

2. The method of claim 1, further wherein the at least one oil comprises plant oil; and wherein the paste-like composition is stirred to achieve a particle size distribution of the magnesium stearate based nanoparticles.

3. The method of claim 1, wherein the separating the magnesium stearate based nanoparticles occurs by at least one method from the following group: filtration, sedimentation, centrifugation, magnetic separation, washing, or any combination thereof.

4. The method of claim 1, further comprising incorporating one or more essentially hydrophilic components into the magnesium stearate based nanoparticles.

5. The method of claim 4, wherein the one or more hydrophilic components comprises at least one active ingredient, marker, passive ingredient, formulation ingredient, or any combination thereof.

6. The method of claim 5, wherein the at least one active ingredient is selected from the group consisting of one or more proteins, peptides, nucleic acids, lipids, amino acids, carbohydrates and derivatives of these aforementioned ingredients, pharmaceutical active ingredients, magnetite, fluorescent markers, and any combination thereof.

7. The method of claim 5, wherein the at least one active ingredient is selected from the group consisting of a protein, a humanized monoclonal antibody, a human monoclonal antibody, a chimeric antibody, an immunoglobulin, fragment, derivative or fraction thereof, a synthetic, semi-synthetic or biosynthetic substance mimicking immunoglobulins or fractions thereof, an antigen binding protein or fragment thereof, a fusion protein or peptide or fragment thereof, a receptor antagonist, an antiangiogenic compound, an intracellular signaling inhibitor, a peptide with a molecular mass equal to or higher than 3 kDa, a ribonucleic acid (RNA), a deoxyribonucleic acid (DNA), a plasmid, a peptide nucleic acid (PNA), a steroid, a corticosteroid, an adrenocorticostatic, an antibiotic, an antidepressant, an antimycotic, a [beta]-adrenolytic, an androgen or antiandrogen, an antianemic, an anabolic, an anesthetic, an analeptic, an antiallergic, an antiarrhythmic, an antiarterosclerotic, an antibiotic, an antifibrinolytic, an anticonvulsive, an anti-inflammatory drug, an anticholinergic, an antihistamine, an antihypertensive, an antihypotensive, an anticoagulant, an antiseptic, an antihemorrhagic, an antimyasthenic, an antiphlogistic, an antipyretic, a beta-receptor antagonist, a calcium channel antagonist, a cell differentiation factor, a chemokine, a chemotherapeutic, a co-enzyme, a cytotoxic agent, a prodrug of a cytotoxic agent, a cytostatic, an enzyme and its synthetic or biosynthetic analogue, a glucocorticoid, a growth factor, a hemostatic, a hormone and its synthetic or biosynthetic analogue, an immunosuppressant, an immunostimulant, a mitogen, a physiological or pharmacological inhibitor of mitogens, a mineralocorticoid, a muscle relaxant, a narcotic, a neurotransmitter, a precursor of neurotransmitter, an oligonucleotide, a peptide, a (para)-sympathomimetic, a (para)-sympatholytic, a sedating agent, a spasmolytic, a vasoconstrictor, a vasodilator, a vector, a virus, a virus-like particle, a virustatic, a wound healing substance and a combination thereof.

8. The method of claim 1, wherein the nanoparticles are biocompatible, biodegradable and possess superparamagnetic properties.

9. The method of claim 1, wherein a sustained intracellular release effect of the nanoparticles is increased compared to a conventional carrier.

10. The method of claim 1, wherein the paste-like composition is low in water and oil fractions.

11. The method of claim 1, wherein the nanoparticles have average particle diameters ranging from approximately 150 nm to approximately 1000 nm.

12. The method of claim 1, wherein the nanoparticles have average particle diameters ranging from approximately 150 nm to approximately 1750 nm.

13. The method of claim 1, wherein the nanoparticles provide intracellular delivery of one or more active ingredients.

14. The method of claim 1, wherein the at least one oil is selected from the group consisting of tocopherol, castor oil, plant oil, and combinations thereof.
